(19) United States
(12) Patent Application Publication
(10) Pub. No.: US 2003/0213002 A1
(43) Pub. Date: Nov. 13, 2003

(54) Extensive myocardial infarction model animal, method for preparation there of, and application for drug screening and regenerative medicine

(76) Inventors: Hiroshi Nakamura, Ube-shi (JP); Masunori Matsuzaki, Ube-shi (JP); Tsutomu Yoshida, Ube-shi (JP)

Correspondence Address: BIRCH STEWART KOLASCH & BIRCH, PO BOX 747, FALLS CHURCH, VA, 22040-0747, US

(21) Appl. No.: 10/141,608

(22) Filed: May 9, 2002

(51) Int. Cl. A01K 67/027

(52) U.S. Cl. 800/3

(57) ABSTRACT

The prior art has problems such as high mortality of model animals due to complication of heart failure, necessity of taking into consideration the effects from other organs for investigation of the mechanism of the disease, being unable to prepare an extensive myocardial infarction model, and others. The invention allows exclusion of the state of heart failure and provides an extensive myocardial infarction model animal which permits to prepare a model of the pathology of extensive myocardial infarction.

The invention is an extensive myocardial infarction model animal, the method for preparation thereof, and the method for screening of drugs and/or genes using the model animal, wherein the heart of a normal animal is extirpated, the left main trunk of the extirpated heart is ligated, the heart after the ligation is heterotopically grafted to the abdomen of another normal animal, and the animal given the heterotopic graft is used as the extensive myocardial infarction model animal. Three weeks after the graft, the model of extensive myocardial infarction pathology is established.

### BACKGROUND OF THE INVENTION

This invention relates to a novel myocardial infarction model animal, particularly to an extensive myocardial infarction model animal that can exclude the pathological state of heart failure and provides an extensive myocardial infarction model, to the method for preparation thereof, and application for drug screening and regenerative medicine.

As a result of Westernization of the lifestyle and eating habits of Japanese, heart diseases, particularly myocardial infarction following coronary occlusion, have been significant medical problems. In general, investigation of the cause and mechanism of diseases including myocardial infarction and efficacy screening tests for verification of drug effect and/or gene transfer on a disease are performed not in normal animals but in disease model animals which are in a certain pathological state.

Such disease model animals have been prepared by surgical or medical treatment of normal animals; for example, diabetic model animals prepared by administration of a causative substance as disclosed in the unexamined published Japanese patent application No.2000-270713, pulmonary infection model animals prepared by inoculation of a bacterial suspension through a tube into the animal body as disclosed in the unexamined published Japanese patent application No.2000-262182, and skin ischemia model animals prepared by surgical treatment as disclosed in the unexamined published Japanese patent application No.1999-308941.

Also myocardial infarction model animals have been prepared by ligation of the coronary artery of animals such as rats, mice, guinea pigs, hamsters and so on.

According to the prior art described above, ligation of the coronary artery which supplies oxygen and nutrients directly to the heart of the animal results in 30 to 50% of the animal death during or after the surgical treatment because of the hard surgical invasion even when the left anterior descending branch is ligated at the middle. Namely decreased pump function of the heart as a result of ligation of the coronary artery (this may cause myocardial infarction) may induce decreased blood flow in major organs such as the lung and the kidney, and finally multiple organ failure (heart failure). The heart failure is the main cause of animal death after the coronary artery ligation.

In the conventional myocardial infarction model animals which may be complicated by heart failure, effects of other organs must be taken into account and thus investigation of the heart itself may be very difficult. It is said that 3 weeks are needed from ligation of the coronary artery till establishment of myocardial infarction, and novel myocardial infarction model animals that are free from the effect of other organs (heart failure is excluded) are necessary to investigate what happens in the heart during this period.

Although an extensive myocardial infarction model may be prepared by ligation of the left main trunk, which is the definite source of the circumflex artery besides the left arterior descending artery, this requires turning up of the left atrium. With the prior art the mortality due to ligation is high and it is impossible to ligate the left anterior descending branch at a site located in the proximity to the middle of the branch. Therefore it is impracticable for the prior art to prepare an extensive myocardial infarction model.

Under these circumstances, the purpose of the invention is to provide extensive myocardial infarction model animals that can exclude heart failure and prepare a model of extensive myocardial infarction, the method for preparation thereof, and a method that permits efficient screening of drugs and/or genes.

To fulfill the above-mentioned purpose, the invention provides an extensive myocardial infarction model animal, the method for preparation thereof, and drug and/or gene screening using the model animal, where the left main trank of the heart of a normal animal is ligated, followed by heterotopic graft of the heart with the ligated coronary artery to the abdomen of another normal animal which is used as an extensive myocardial infarction model animal.

This invention is an extensive myocardial infarction model animal, the method for preparation thereof, and drug and/or gene screening using the model animal, where the above-mentioned surgical treatment for ligation of the coronary artery comprises a process to extirpate the heart from a normal animal and a process to ligate the left main trunk of the extirpated heart.

This invention is an extensive myocardial infarction model animal, the method for preparation thereof, and drug and/or gene screening using the model animal, where above-mentioned ligation of the left main trunk is performed by turning up of the left atrium, followed either by (1) single ligation of the left main trunk (LMT) or by (2) simultaneous ligation of the left anterior descending artery (LAD) in the proximal segment and the left circumflex artery (CX) in the proximal segment(equivalent to LMT ligation).

This invention is an extensive myocardial infarction model animal, the method for preparation thereof, and drug and/or gene screening using the model animal, where myocardial infarction is established after a specified time, for example after 3 weeks, after heterotopic graft of the heart with the left coronary artery ligated in the LMT into the abdomen of another normal animal.

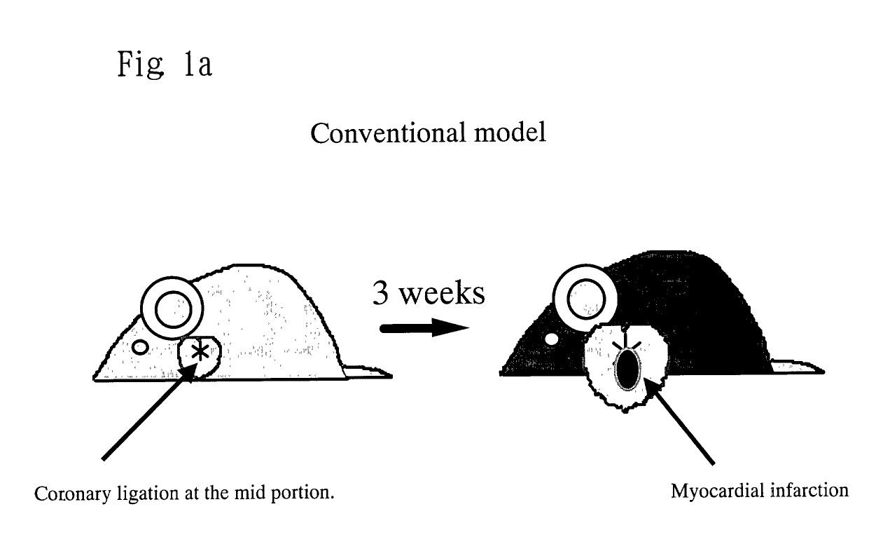

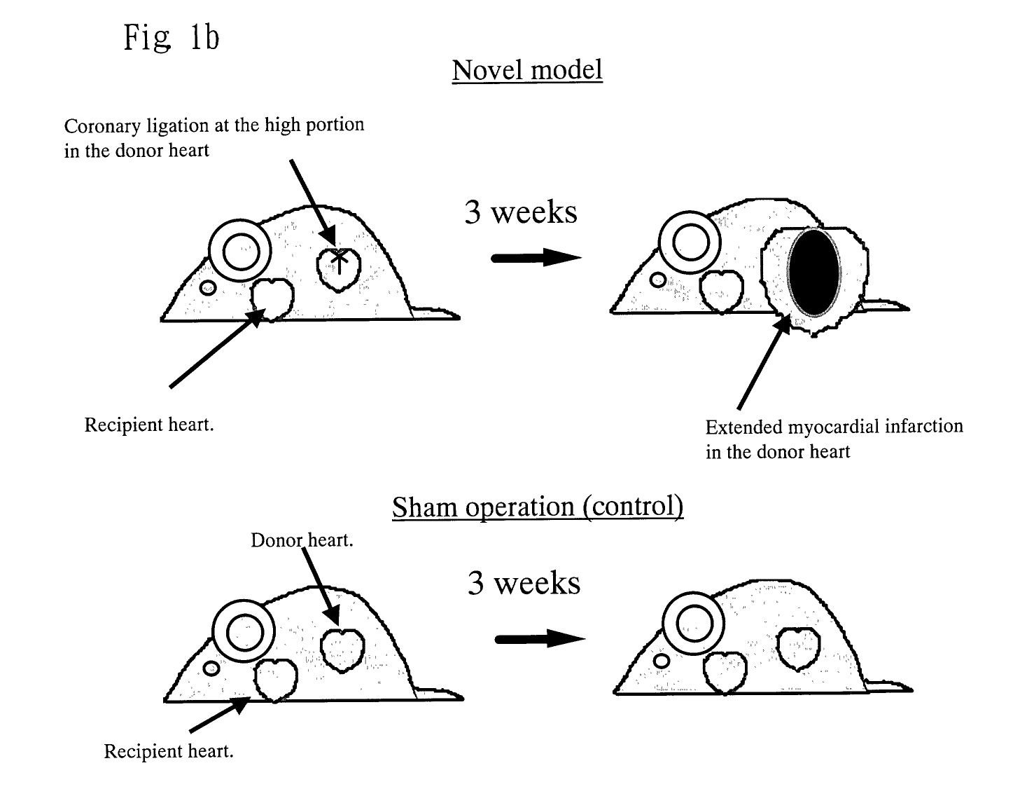

### BRIEF DESCRIPTION OF THE DRAWINGS

[FIG. 1] A figure showing the idea of the Example of the extensive myocardial infarction model animal, the method for preparation thereof, and application for drug screening and regenerative medicine, in comparison with the prior art.

(FIG. 1a) A scheme of conventional model for myocardial infarction. This model is highly complicated by subsequent heart failure and cardiac death after coronary ligation. The mortality rate after the operation is 30-50% in this model.

(FIG. 1b) An conceptual scheme of novel cardiac transplantation and simultaneous coronary artery ligation. Coronary ligation is performed at the high portion of the donor heart. Therefor, extended myocardial infarction occurs in the donor heart. Although it takes about one hour for this opeartion, the survival rate is quietly high comparing with the conventional MI model(≠95%). Lower panel shows a scheme of the control group(sham operation).

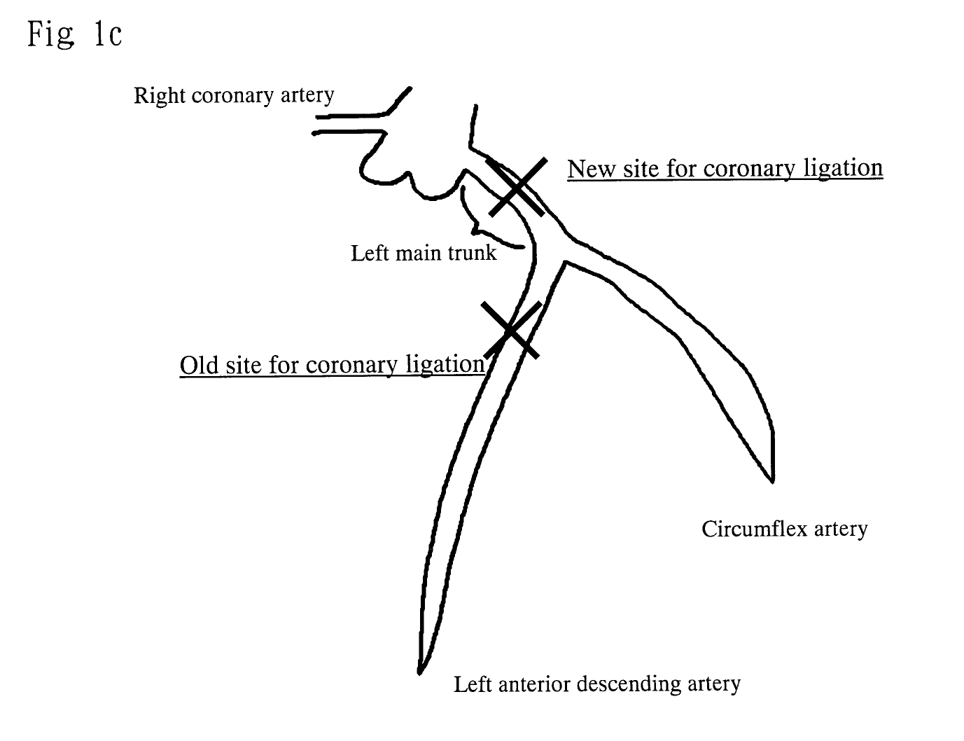

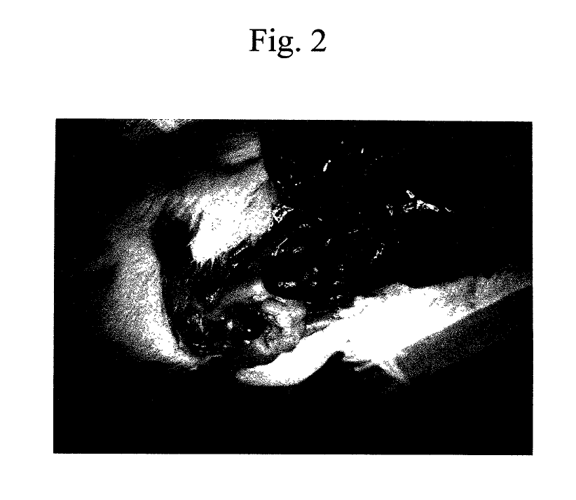

(FIG. 1c) An anatomical scheme of the ligation site of the coronary artery. In the conventional model, the site for the coronary ligation is up to the mid portion of left anterior discendent artery. Because, the ligation in the more proximal site results in lethal for the animal. In the contrary, it is possible to ligate the coronary artery at more proximal site with highly survival rate( 95%).

[FIG. 2] A photograph showing an example of the extensive myocardial infarction model animal of the invention where the model of extensive myocardial infarction is established by ligation of the LMT.

### EXAMPLES

The extensive myocardial infarction model animals of the invention are not particularly specified as far as they permit investigation of the cause and mechanism of onset and verification of drug and/or gene transfer's effect on myocardial infarction, and exemplified by rats, mice, guinea pigs, hamsters and so on, among which rats are preferable.

The left coronary artery of the heart of a normal animal is ligated in the proximal segment, followed by heterotopic graft of the heart with the ligated coronary artery to the abdomen of another normal animal, and the animal that has received heterotopic graft of the heart is used as the extensive myocardial infarction model animal. Preferably, the heart of a normal animal is extirpated, and the left coronary artery of the extirpated heart is ligated in the proximal segment, followed by heterotopic graft of the heart to the abdomen of another normal animal. Rejection episodes following heterotopic graft of the heart can totally be avoided by using an inbred animal as the recipient animal of heterotopic heart graft.

The extensive myocardial infarction model animals of the invention are model animals having two hearts, a normal heart and the heart with the left main trank (LMT) ligation, where, according to the invention, heart failure, a multifactorial phenomenon that is unnecessary for investigation of the cause and mechanism of myocardial infarction can be excluded. Because the recipient (the animal that receives the organ) is a normal animal, the drug and/or gene transfer's effect on myocardial infarction can be recognized clearly, which permits efficient screening tests of drug and/or gene transfer's efficacy. With an adequate technique of heterotopic heart graft, extensive myocardial infarction model animals would be supplied constantly, the mortality due to surgical treatment would be less than 5%, and the rate of successful heart graft would be about 100%.

The invention is able to prepare a model of extensive myocardial infarction, which was impossible with the prior art because of the excessively severe technical invasion, by ligation in the proximal segment of the left coronary artery, for example by turning up of the left atrium followed either by ligation of the main trunk of the left coronary artery or by simultaneous ligation of the left anterior descending branch in the proximal segment and the left circumflex branch in the proximal segment, and heterotopic graft of the heart to the abdomen of another normal animal.

The site of ligation is not limited to the above-mentioned sites, and it is possible to prepare a myocardial infarction model of an arbitrary size by changing the proximity of the site of ligation. With the prior art ligation was possible only up to the middle of the left anterior descending branch, whereas with the invention, it is possible to obtain an extensive myocardial infarction model at a probability as high as 95% or more even when the two coronary arteries are ligated in the proximal segment.

In the invention, after a specified time, for example after 3 weeks, after heterotopic graft of the heart with the LMT ligation to the abdomen of another normal animal, the animal that has received heterotopic heart graft is used as the extensive myocardial infarction model animal. After 3 weeks the heart transplanted is extirpated and used for investigation of the cause and mechanism of onset. The recipient animal may be used for verification of the effect of a drug and/or gene transfer on myocardial infarction in progress when the animal is treated with the drug during the 3-week period, and/or with gene transfer during the operation.

The time required for establishment of myocardial infarction after coronary ligation varies according to the animal species used or the level of proximity of the ligation site, and is not limited to 3 weeks.

Whether myocardial infarction has been established can be judged based on changes in individual characteristics, such as body weight or urinary volume, by chemical tests, etc., and the invention does not limit the measures and methods for judgment on establishment of myocardial infarction.

The extensive myocardial infarction model animal of the invention, or the extensive myocardial infarction model animal prepared by the method of the invention, is free of heart failure and provides a model of extensive myocardial infarction. Thus this animal may be used for efficient drug and/or gene screening.

In the following, the example of the invention is explained by using the drawings.

FIG. 1 illustrates the idea of the invention by the example of the extensive myocardial infarction model animal, the method for preparation thereof, and drug screening by using the animal in comparison with the prior art. In FIG. 1, FIG. 1b is the model prepared by the prior art, FIG. 1b is the anatomical scheme for the coronay ligation, in this model, and FIG. 1c is the novel prepared by the invention.

The conventional model shown in FIG. 1a is obtained by ligation of the left anterior descending branch at the middle given to the heart of a normal rat with the intention of obtaining a myocardial infarction model animal after 3 weeks. With this prior art, 30 to 50% of the rats died of heart failure complicated at a high incidence in spite of ligation given at the middle of the left anterior descending artery. So this is the limitation of the conventional model. If they tried to ligate more proximal site of the coronary artery to get more extended myocardial infarction, they will loss almost all of the aminals (FIG. 1a). In FIG. 1b of the novel model, the lower column shows the state of the rat given heterotopic graft of the heart from another normal rat which is not illustrated , and the upper column shows the state of the rat given heterotopic graft of the heart with the left coronary artery ligated at the origin from another normal rat which is not illustrated. Comparison of these two different states eliminates the effect of heterotopic heart graft, and such a procedure is common in investigation of the cause and mechanism of diseases or in drug and/or gene screening in disease model animals.

The novel model shown in FIG. 1c where the detailed illustration was omitted is obtained by extirpation of the heart from a different normal rat not illustrated, ligation of the left coronary artery of the extirpated heart at the origin, and heterotopic graft of the heart to the abdomen of a normal rat with the intention of obtaining an extensive myocardial infarction model animal after 3 weeks. In the extensive myocardial infarction model animal of the example, the arterial system and the venous system of the heart after ligation of the coronary artery were linked with the artery and with the vein, respectively, of the abdomen of the recipient, and thus the model animal has two hearts arranged in a row, a normal heart and a pathological heart with myocardial infarction.

In the example, the mortality after 3 weeks was less than 5% in spite of the high proximity level of ligation of the left coronary artery at the origin, that is, we could prepare extensive myocardial infarction model animals at a high percentage of success.

FIG. 2 is a photograph of an example of the extensive myocardial infarction model animals of the invention prepared by ligation of the LMT; the normal rat laparotomized 3 weeks after heterotopic graft of the heart with the LMT ligation. In FIG. 2, the right-hand is the caudal side and the left-hand is the cranial side of the recipient rat. The liver is on the left side and the transplanted heart is in the middle. The photograph shows that the transplanted heart became larger with its brownish right half, which indicates that the extensive myocardial infarction was established.

With the invention, heart failure can be excluded and the model of extensive myocardial infarction can be obtained at a probability as high as 95% or more. Use of the extensive myocardial infarction model animal of this invention permits efficient investigation of the cause and mechanism of myocardial infarction and also efficient screening of drugs and/or genes for myocardial infarction.

The example of this invention has been explained, and it is apparent that various changes are allowable in the mode and details without deviation from the spirit and the scope of the invention.

For example, the above-mentioned example is ligation of LMT, but the proximity level of the ligation site depends on the severity of myocardial infarction required, and thus does not limit the invention at all.

The invention has an effect to exclude heart failure, a multifactorial phenomenon unnecessary for investigation of the cause and mechanism of myocardial infarction, as the result of heterotopic graft of the heart with the LMT ligation to the abdomen of another normal animal. The invention has an additional effect to prepare an extensive myocardial infarction model which was impossible with the prior art because of the very hard surgical invasion but possible with the invention, for example, by ligation of the left coronary arterial main trunk followed by heterotopic graft of the heart to the abdomen of another normal animal. In addition, the extensive myocardial infarction model of this invention where heart failure can be excluded permits efficient screening tests of drugs and/or genes for myocardial infarction, a significant problem in medicine. Thus the industrial applicability of the invention is very great.

What is claimed is

1) an extensive myocardial infarction model animal characterized in that the left main trunk of the heart of a normal animal is ligated, followed by heterotopic graft of the heart with the ligated coronary artery to the abdomen of another normal animal, and the animal given heterotopic heart graft is used as the myocardial infarction model animal:

2) An extensive myocardial infarction model animal according to claim (1), characterized in that the ligation of the coronary artery comprises a process of extirpation of the heart of a normal animal and a process of ligation of the left main trunk of the extirpated heart.

3) An extensive myocardial infarction model animal according to claim (1) or claim (2), characterized in that the above-mentioned ligation of the left coronary artery in the proximal segment consists in turning up of the left atrium, followed either by (1) single ligation of the left coronary arterial main trunk (LMT) or by (2) simultaneous ligation of the left anterior descending artery (LAD) in the proximal segment and the left circumflex artery (CX) in the proximal segment.

4) A method for preparation of an extensive myocardial infarction model animal characterized in that the LMT of the heart of a normal animal is ligated in the proximal segment, followed by heterotopic graft of the heart with the ligated coronary artery to the abdomen of another normal animal to prepare a pathological state of myocardial infarction after a specified time after the heterotopic graft.

5) A method for preparation of an extensive myocardial infarction model animal according to claim (4), characterized in that the ligation of the coronary artery comprises a process of extirpation of the heart of a normal animal and a process of LMT ligation of the extirpated heart.

6) A method for preparation of an extensive myocardial infarction model animal according to claim (4) or claim (5), characterized in that the above-mentioned ligation of the LMT consists in turning up of the left atrium, followed either by (1) single ligation of the left coronary arterial main trunk (LMT) or by (2) simultaneous ligation of the left anterior descending artery (LAD) in the proximal segment and the left circumflex artery (CX) in the proximal segment.

7) A method for preparation of an extensive myocardial infarction model animal according to claim (4) or claim (5) or claim (6), characterized in that the above-mentioned specified time is 3 weeks or longer.

8) A method for screening of drugs and/or genes using the extensive myocardial infarction model animal described in any of claim (1) to claim (3) or the extensive myocardial infarction model animal prepared by the method described in any of claim (4) to claim (7).
